(19) United States
(12) Patent Application Publication
(10) Pub. No.: US 2020/0148758 A1
(43) Pub. Date: May 14, 2020

(54) THERAPEUTIC COMBINATIONS COMPRISING EOSINOPHIL-DEPLETING ANTIBODIES AND USES THEREOF

(71) Applicant: VAL-CHUM, LIMITED PARTNERSHIP, Montreal (CA)

(72) Inventors: John STAGG, Montreal (CA); Marjorie-Allison BERGERON, Montreal (CA)

(21) Appl. No.: 16/621,532

(22) PCT Filed: Jun. 12, 2018
(86) PCT No.: PCT/CA2018/050696

§ 371 Date: Dec. 11, 2019

Related U.S. Application Data
(60) Provisional application No. 62/518,119, filed on Jun. 12, 2017.

(51) Int. Cl. C07K 16/24; C07K 16/28; A61P 35/00

(52) U.S. Cl. CPC C07K 16/244; C07K 16/2818; A61K 2039/507; C07K 16/2803; C07K 16/2896; A61P 35/00

(57) ABSTRACT

Anti-cancer immune-stimulating therapies have shown inconsistent results when used in humans. The present disclosure provides methods of using eosinophil-depleting agents to increase the therapeutic activity of anti-cancer immune-stimulating therapies. The present disclosure also provides methods of identifying subjects susceptible to respond to anti-cancer immune-stimulating therapies based on their ability to reduce the number of tumor regulatory T cells.

### CROSS-REFERENCE TO RELATED APPLICATIONS AND DOCUMENTS

This application claims priority from U.S. provisional patent application 62/518,119 filed on Jun. 12, 2018 and herewith incorporated in its entirety.

### TECHNOLOGICAL FIELD

The present disclosure concerns methods for increasing the therapeutic activity of anti-cancer immune-stimulating agents as well as method for identifying responders to anti-cancer stimulating agents.

### BACKGROUND

Anti-cancer immune-stimulating therapies provide additional therapeutic alternatives to cancer patients. Despite the fact that anti-cancer immune-stimulating therapies exhibited promising results, their widespread use is not possible as many patients fails to respond appropriately to such therapies (Seymour et al., 2017; Huang et al., 2017).

There is thus a need to provide improved anti-cancer immune-stimulating therapies by either combining them with additional therapeutic agents or by being to identify responders to the anti-cancer immune-stimulating therapies.

### BRIEF SUMMARY

The present disclosure provides improved anti-cancer immune-stimulating therapies. In an embodiment, the present disclosure provides methods of using an eosinophil-depleting agent in combination with an anti-cancer immune-stimulating agent. The eosinophil-depleting agent can be a known eosinophil-depleting agent or it could have been identified with the screening method also disclosed herewith. In another embodiment, the present disclosure provides a method for identifying cancer subjects responding to the anti-cancer immune-stimulating agent based on their ability to substantially maintain the levels of tumoral regulatory T cells.

In a first aspect, the present disclosure provides a method of increasing the therapeutic activity of an anti-cancer immune-stimulating agent in a subject having a tumor. Broadly, the method comprises administrating an eosinophil-depleting therapeutic agent prior to, concomitantly or after having administered the anti-cancer immune-stimulating agent to the cancer subject thereby increasing the therapeutic activity of the anti-cancer immune-stimulating agent. Upon administration to the subject, the eosinophil-depleting agent is capable of at least partially depleting tumor-specific eosinophils. In addition, upon administration to the subject, the anti-cancer immune-stimulating agent lacks the ability to substantially reduce the number of tumor-associated regulatory T cells. In an embodiment, the eosinophil-depleting agent is administered to the subject prior to or concomitantly with the anti-cancer stimulating agent. In an embodiment, the method can further comprise determining if the anti-cancer immune-stimulating agent lacks the ability to substantially reduce the number of tumor-associated regulatory T cells in the subject. In still a further embodiment, the method can further comprise administering the eosinophil-depleting agent and/or the anti-cancer immune stimulating agent when the anti-cancer immune-stimulating agent has been determined to lack the ability to substantially reduce the number of tumor-associated regulatory T cells. In an embodiment, the eosinophil-depleting agent is an antibody, such as, for example, a monoclonal antibody. In additional embodiments, the eosinophil-depleting agent can be an anti-growth factor antibody, an anti-cytokine antibody, an anti-cell surface antibody and/or an anti-prostaglandin receptor antibody. In yet another embodiment, the eosinophil-depleting agent can be an anti-interleukin antibody, such as, for example, an anti-interleukin-5 antibody. In a further embodiment, the anti-cancer immune-stimulating agent is an antagonistic (monoclonal) antibody. In yet another embodiment, the antagonistic antibody can be specific for an immune check-point. In specific embodiments, the anti-cancer immune stimulating agent is an anti-cytotoxic T-lymphocyte associated protein 4 (CTLA-4) antibody, an anti-programmed cell death 1 (PD-1) antibody and/or an anti-T cell immunoglobulin and mucin domain 3 (TIM-3) antibody. In additional embodiments, the anti-cancer immune-stimulating agent is an agonistic (monoclonal) antibody. For example, the agonistic antibody can be an anti-cancer immune-stimulating agent is an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In a further embodiment, the subject has a predisposition to the cancer, has the cancer or was previously afflicted with the cancer. In yet another embodiment, the cancer is at least one of a lung cancer, a breast cancer, a gastro-intestinal cancer, a head and neck cancer, an ovarian cancer, an adrenal gland cancer, a thyroid cancer, a biliary cancer, a bladder cancer, a urinary tract cancer, an endometrial cancer, a prostate cancer, a liver cancer, a mouth cancer, a lymphocyte cancer, a rectal cancer and/or a muscle cancer. In still a further embodiment, the tumor is a solid tumor or a liquid tumor. In still another embodiment, the subject can be a human.

In a second aspect, the present disclosure provides a method of treating or alleviating the symptoms of a cancer in a subject having a tumor. Broadly, the method comprises administering an eosinophil-depleting agent and an anti-cancer immune-stimulating agent thereby treating or alleviating the symptoms of the cancer in the subject. Upon administration to the subject, the eosinophil-depleting agent is capable of at least partially depleting tumor-specific eosinophils. In addition, and still upon administration to the subject, the anti-cancer immune-stimulating agent lacks the ability to substantially reduce the number of tumor-associated regulatory T cells. In an embodiment, the eosinophil-depleting agent is administered to the subject prior to or concomitantly with the anti-cancer stimulating agent. In an embodiment, the method can further comprise determining if the anti-cancer immune-stimulating agent lacks the ability to substantially reduce the number of tumor-associated regulatory T cells in the subject. In still a further embodiment, the method can further comprise administering the eosinophil-depleting agent and/or the anti-cancer immune stimulating agent when the anti-cancer immune-stimulating agent has been determined to lack the ability to substantially reduce the number of tumor-associated regulatory T cells. In an embodiment, the eosinophil-depleting agent is an antibody, such as, for example, a monoclonal antibody. In additional embodiments, the eosinophil-depleting agent can be an anti-growth factor antibody, an anti-cytokine antibody, an anti-cell surface antibody and/or an anti-prostaglandin receptor antibody. In yet another embodiment, the eosinophil-depleting agent can be an anti-interleukin antibody, such as, for example, an anti-interleukin-5 antibody. In a further embodiment, the anti-cancer immune-stimulating agent is an antagonistic (monoclonal) antibody. In yet another embodiment, the antagonistic antibody can be specific for an immune check-point. In specific embodiments, the anti-cancer immune stimulating agent is an anti-cytotoxic T-lymphocyte associated protein 4 (CTLA-4) antibody, an anti-programmed cell death 1 (PD-1) antibody and/or an anti-T cell immunoglobulin and mucin domain 3 (TIM-3) antibody. In additional embodiments, the anti-cancer immune-stimulating agent is an agonistic (monoclonal) antibody. For example, the agonistic antibody can be an anti-cancer immune-stimulating agent is an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In a further embodiment, the subject has a predisposition to the cancer, has the cancer or was previously afflicted with the cancer. In yet another embodiment, the cancer is at least one of a lung cancer, a breast cancer, a gastro-intestinal cancer, a head and neck cancer, an ovarian cancer, an adrenal gland cancer, a thyroid cancer, a biliary cancer, a bladder cancer, a urinary tract cancer, an endometrial cancer, a prostate cancer, a liver cancer, a mouth cancer, a lymphocyte cancer, a rectal cancer and/or a muscle cancer. In still a further embodiment, the tumor is a solid tumor or a liquid tumor. In still another embodiment, the subject can be a human.

In a third aspect, the present disclosure provides an eosinophil-depleting agent for increasing the therapeutic activity of an anti-cancer immune-stimulating agent in a subject having a tumor. In such aspect, the eosinophil-depleting agent is capable of at least partially depleting tumor-specific eosinophils and the anti-cancer immune-stimulating agent lacks the ability to substantially reduce the number of the tumor-associated regulatory T cells. In an embodiment, the eosinophil-depleting agent is adapted for administration to the subject prior to or concomitantly with the anti-cancer immune-stimulating agent. In still another embodiment, the anti-cancer immune-stimulating agent has been determined to lack the ability to reduce the number of tumor-associated regulatory T cells. In another embodiment, the eosinophil-depleting agent is an antibody, such as, for example, a monoclonal antibody. In a further embodiment, the eosinophil-depleting agent is an anti-growth factor antibody, an anti-cytokine antibody, an anti-cell surface antibody and/or an anti-prostaglandin receptor antibody. The eosinophil-depleting agent can be an anti-interleukin antibody, such as, for example an anti-interleukin-5 antibody. In another embodiment, the anti-cancer immune-stimulating agent is an antagonistic antibody, such as, for example, an antagonistic monoclonal antibody. The antagonistic antibody can be specific for an immune check-point, such as, for example, an anti-cytotoxic T-lymphocyte associated protein 4 (CTLA-4) antibody, an anti-programmed cell death 1 (PD-1) antibody and/or an anti-T cell immunoglobulin and mucin domain 3 (TIM-3) antibody. In another embodiment, the anti-cancer immune-stimulating agent can be an agonistic antibody, such as, an agonistic monoclonal antibody. The agonistic antibody can be for example, an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In an embodiment, the subject has a predisposition to the cancer, has the cancer or was previously afflicted with the cancer. In yet another embodiment, the cancer can be at least one of a lung cancer, a breast cancer, a gastro-intestinal cancer, a head and neck cancer, an ovarian cancer, an adrenal gland cancer, a thyroid cancer, a biliary cancer, a bladder cancer, a urinary tract cancer, an endometrial cancer, a prostate cancer, a liver cancer, a mouth cancer, a lymphocyte cancer, a rectal cancer and/or a muscle cancer. In still another embodiment, the tumor can be a solid tumor or a liquid tumor. In a further embodiment, the subject is a human.

In a fourth aspect, the present disclosure provides an eosinophil-depleting agent for treating or alleviating the symptoms of a cancer in a subject having a tumor. In such aspect, the eosinophil-depleting agent is capable of at least partially depleting tumor-specific eosinophils, the eosinophil-depleting agent is for use in combination with an anti-cancer immune-stimulating agent, and the anti-cancer immune-stimulating agent lacks the ability to substantially reduce the number of the tumor-associated regulatory T cells. In an embodiment, the eosinophil-depleting agent is adapted for administration to the subject prior to or concomitantly with the anti-cancer immune-stimulating agent. In still another embodiment, the anti-cancer immune-stimulating agent has been determined to lack the ability to reduce the number of tumor-associated regulatory T cells. In another embodiment, the eosinophil-depleting agent is an antibody, such as, for example, a monoclonal antibody. In a further embodiment, the eosinophil-depleting agent is an anti-growth factor antibody, an anti-cytokine antibody, an anti-cell surface antibody and/or an anti-prostaglandin receptor antibody. The eosinophil-depleting agent can be an anti-interleukin antibody, such as, for example an anti-interleukin-5 antibody. In another embodiment, the anti-cancer immune-stimulating agent is an antagonistic antibody, such as, for example, an antagonistic monoclonal antibody. The antagonistic antibody can be specific for an immune check-point, such as, for example, an anti-cytotoxic T-lymphocyte associated protein 4 (CTLA-4) antibody, an anti-programmed cell death 1 (PD-1) antibody and/or an anti-T cell immunoglobulin and mucin domain 3 (TIM-3) antibody. In another embodiment, the anti-cancer immune-stimulating agent can be an agonistic antibody, such as, for example, an agonistic monoclonal antibody. The agonistic antibody can be an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In an embodiment, the cancer subject has a predisposition to the cancer, has the cancer or was previously afflicted with the cancer. In yet another embodiment, the cancer can be at least one of a lung cancer, a breast cancer, a gastro-intestinal cancer, a head and neck cancer, an ovarian cancer, an adrenal gland cancer, a thyroid cancer, a biliary cancer, a bladder cancer, a urinary tract cancer, an endometrial cancer, a prostate cancer, a liver cancer, a mouth cancer, a lymphocyte cancer, a rectal cancer and/or a muscle cancer. In still another embodiment, the tumor can be a solid tumor or a liquid tumor. In a further embodiment, the subject is a human.

In a fifth aspect, the present disclosure provides an anti-cancer immune stimulating agent for treating or alleviating the symptoms of a cancer in a subject. In such aspect, the anti-cancer immune-stimulating agent is for use in combination with an eosinophil-depleting agent, the anti-cancer immune-stimulating agent lacks the ability to substantially reduce the number of the tumor-associated regulatory T cells and the eosinophil-depleting agent is capable of at least partially depleting tumor-specific eosinophils. In an embodiment, the eosinophil-depleting agent is adapted for administration to the subject prior to or concomitantly with the anti-cancer immune-stimulating agent. In still another embodiment, the anti-cancer immune-stimulating agent has been determined to lack the ability to reduce the number of tumor-associated regulatory T cells. In another embodiment, the eosinophil-depleting agent is an antibody, such as, for example, a monoclonal antibody. In a further embodiment, the eosinophil-depleting agent is an anti-growth factor antibody, an anti-cytokine antibody, an anti-cell surface antibody and/or an anti-prostaglandin receptor antibody. The eosinophil-depleting agent can be an anti-interleukin antibody, such as, for example an anti-interleukin-5 antibody. In another embodiment, the anti-cancer immune-stimulating agent is an antagonistic antibody, such as, for example, an antagonistic monoclonal antibody. The antagonistic antibody can be specific for an immune check-point, such as, for example, an anti-cytotoxic T-lymphocyte associated protein 4 (CTLA-4) antibody, an anti-programmed cell death 1 (PD-1) antibody and/or an anti-T cell immunoglobulin and mucin domain 3 (TIM-3) antibody. In another embodiment, the anti-cancer immune-stimulating agent can be an agonistic antibody, such as, for example, an agonistic monoclonal antibody. The agonistic antibody can be an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In an embodiment, the cancer subject has a predisposition to the cancer, has the cancer or was previously afflicted with the cancer. In yet another embodiment, the cancer can be at least one of a lung cancer, a breast cancer, a gastro-intestinal cancer, a head and neck cancer, an ovarian cancer, an adrenal gland cancer, a thyroid cancer, a biliary cancer, a bladder cancer, a urinary tract cancer, an endometrial cancer, a prostate cancer, a liver cancer, a mouth cancer, a lymphocyte cancer, a rectal cancer and/or a muscle cancer. In still another embodiment, the tumor can be a solid tumor or a liquid tumor. In a further embodiment, the subject is a human.

In a sixth aspect, the present disclosure provides a pharmaceutical combination for treating or alleviating the symptoms of cancer in a subject having a tumor, the pharmaceutical combination comprising (i) an eosinophil-depleting agent capable of at least partially depleting tumor-specific eosinophils and (ii) an anti-cancer immune-stimulating agent lacking the ability to substantially reduce the tumor-associated regulatory T cells. In an embodiment, the eosinophil-depleting agent is adapted for administration to the subject prior to or concomitantly with the anti-cancer immune-stimulating agent. In still another embodiment, the anti-cancer immune-stimulating agent has been determined to lack the ability to reduce the number of tumor-associated regulatory T cells. In another embodiment, the eosinophil-depleting agent is an antibody, such as, for example, a monoclonal antibody. In a further embodiment, the eosinophil-depleting agent is an anti-growth factor antibody, an anti-cytokine antibody, an anti-cell surface antibody and/or an anti-prostaglandin receptor antibody. The eosinophil-depleting agent can be an anti-interleukin antibody, such as, for example an anti-interleukin-5 antibody. In another embodiment, the anti-cancer immune-stimulating agent is an antagonistic antibody, such as, for example, an antagonistic monoclonal antibody. The antagonistic antibody can be specific for an immune check-point, such as, for example, an anti-cytotoxic T-lymphocyte associated protein 4 (CTLA-4) antibody, an anti-programmed cell death 1 (PD-1) antibody and/or an anti-T cell immunoglobulin and mucin domain 3 (TIM-3) antibody. In another embodiment, the anti-cancer immune-stimulating agent can be an agonistic antibody, such as, for example, an agonistic monoclonal antibody. The agonistic antibody can be an anti-TNF receptor superfamily member 9 (TNFRSF9) antibody, an anti-TNF receptor superfamily member 4 (TNFRSF4) antibody and/or an anti-TNF receptor superfamily member 18 (TNFRSF18) antibody. In an embodiment, the cancer subject has a predisposition to the cancer, has the cancer or was previously afflicted with the cancer. In yet another embodiment, the cancer can be at least one of a lung cancer, a breast cancer, a gastro-intestinal cancer, a head and neck cancer, an ovarian cancer, an adrenal gland cancer, a thyroid cancer, a biliary cancer, a bladder cancer, a urinary tract cancer, an endometrial cancer, a prostate cancer, a liver cancer, a mouth cancer, a lymphocyte cancer, a rectal cancer and/or a muscle cancer. In still another embodiment, the tumor can be a solid tumor or a liquid tumor. In a further embodiment, the subject is a human.

In a seventh aspect, the present disclosure provides a method for identifying a therapeutic agent capable of increasing the therapeutic activity of an anti-cancer immune-stimulating agent and/or of treating or alleviating the symptoms of a cancer subject having a tumor. Broadly, the method comprises: a) determining the ability of a test agent to deplete eosinophils; b) providing the ability of a control agent to deplete eosinophils; and c) characterizing the test agent as being useful for increasing the therapeutic activity of the anti-cancer immune-stimulating agent and/or for treating or alleviating the symptoms of the cancer subject when the test agent has been determined to be able to deplete more eosinophils than the control agent. In an embodiment, step c) further comprises characterizing the test agent as lacking the usefulness for increasing the therapeutic activity of the anti-cancer immune-stimulating agent and/or for treating or alleviating the symptoms of the cancer subject when the test agent has been determined not to be able to deplete more eosinophils than the control agent. In another embodiment, step a) and/or b) is(are) conducted in a non-human animal. In still a further embodiment, the non-human animal bears a tumor. In yet another embodiment, the eosinophils are tumor-associated eosinophils.

In an eighth aspect, the present disclosure provides a method to determining the usefulness of an anti-cancer immune-stimulating agent for treating or alleviating the symptoms of a cancer in a subject having a tumor. Broadly, the method comprises a) determining the number of regulatory T cells in the tumor either prior to contacting the anti-cancer immune-stimulating agent with the tumor or in the presence of a control agent; b) determining the number of regulatory T cells ability in the tumor after contacting the tumor with the anti-cancer immune-stimulating; and c) characterizing the anti-cancer immune-stimulating agent as being useful for treating or alleviating the symptoms of the subject when the anti-cancer immune-stimulating agent has been characterized as lacking the ability to substantially decrease the number of regulatory T cells in the tumor, when compared to the number of regulatory T cells determined in step a). In an embodiment, the method further comprises administering the anti-cancer immune-stimulating agent to the cancer subject when step c) characterizes the anti-cancer stimulating agent as being useful for treating or alleviating the symptoms of the cancer subject. In yet another embodiment, the anti-cancer immune stimulating agent is as described herein. In a further embodiment, the method further comprises administering an eosinophil-depleting agent to the cancer patient when step c) characterizes the anti-cancer stimulating agent as being useful for treating or alleviating the symptoms of the cancer subject. In yet another embodiment, the eosinophil-depleting agent is as described herein. In yet another embodiment, the eosinophil-depleting agent and the anti-cancer immune-stimulating agent are administered according to the method is as described herein. In yet another embodiment, the cancer subject is as described herein.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A to C illustrate that anti-CTLA-4 monoclonal antibody (mAb) therapy of MC38 subcutaneous tumors is enhanced in eosinophil-deficient mice. Results as shown for wild-type (VVT) and eosinophil-deficient (Δdbl-Gata) C57BI/6 mice treated with a control immunoglobulin (clg; clone 2A3) (FIG. 1A) or anti-CTLA-4 mAb (αCTLA-4; clone 4F10) (FIG. 1B). Mean tumor sizes and standard errors of 10 mice per group (provided in mm²) are shown in function of days post-injection of tumor cells. When significant, Mann-Whitney P value at end-point is shown (FIG. 1B). FIG. 1C illustrates that anti-CTLA-4 mAb therapy of CT26 subcutaneous tumors is enhanced in eosinophil-deficient mice. Results as shown for WT and eosinophil-deficient (Gata1-tm6Sho) BALB/c mice treated with control Ig (clone 2A3) or anti-CTLA-4 mAb (clone 9H10). Survival of mice (11 mice per group) is shown in function of days post-injection of tumor cells. Log-rank P value less than 0.05 between WT and eosinophil-deficient mice treated with anti-CTLA-4 is depicted (*).

FIGS. 2A to G illustrate that anti-PD-1 (clone RMP1-14), anti-CD137 mAb (clone 3H3) and anti-TIM-3 (clone RMT3-23) therapy of subcutaneous MC38 tumors is enhanced in eosinophil-deficient mice. Results as shown for wild-type (WT—FIGS. 2A to 2C) and eosinophil-deficient (Δdbl GATA—FIGS. 2D to 2F) mice treated with anti-PD-1 (FIGS. 2A and D), anti-CD137 (FIGS. 2B and E) mAb and anti-TIM-3 (FIGS. 2C and F). Results are presented for each of the treated mice (one line per mouse) as the mean tumor size (provided in mm²) in function of days post-injection of tumor cells. The number of tumor-free mice at day 40 is indicated on each panel. FIG. 2G illustrates that anti-PD-1 therapy (αPD-1; clone RMP1-14) of subcutaneous MC38 tumors is enhanced in eosinophil-deficient (Δdbl GATA) mice compared to wild type (WT) mice. Survival of mice (33 per group from 4 pooled independent experiments) is shown in function of days post-injection of tumor cells. Log-rank P value less than 0.01 between WT and eosinophil-deficient mice is depicted (**).

FIGS. 3A and 3B illustrate that blocking interleukin(IL)-5 mAb enhances the activity of anti-CTLA-4 mAb in stimulating anti-tumor T cells within tumors. Results are provided as (FIG. 3A) the mean number of tumor-specific T cells and (FIG. 3B) the mean percentage of tumor-positive T cells (when compared to the total of CD8+ cells) in control-treated (control), anti-IL-5 treated (aIL-5), anti-CTLA-4 treated (aCTLA-4) or anti-IL-S/anti-CTLA4-treated (combo) animals (*p<0.05; **p<0.01 compared to control).

FIGS. 4A to 4C illustrate that eosinophil-associated genes are associated with poor prognosis in cancer patients. Results are shown for high and low expression levels of (FIG. 4A) IL-5, (FIG. 4B) eotaxin and (FIG. 4C) CCR3 in function of overall survival (in months) in lung non-small cell adenocarcinoma.

FIGS. 5A to D illustrate that anti-CTLA-4 therapy (with clone 9H10) is enhanced in subcutaneous CT26 tumors but not in subcutaneous MC38 tumors. Results as shown for wild-type (WT—FIGS. 5A and B) and eosinophil-deficient (Δbl GATA—FIGS. 5C and D) syngeneic mice treated with anti-CTLA-4 mAb and bearing CT26 (FIGS. A and C) or MC38 (FIGS. B and D) tumors. Results are presented for each of the treated mice (one line per mouse) as the mean tumor size (provided in mm²) in function of days post-injection of tumor cells. The number of tumor-free mice at day 60 is indicated on each panel.

FIGS. 6A and B illustrate that anti-CTLA-4 mAb therapy with clone 9H10 maintains tumor-associated regulatory T cell (Treg) levels in CT26 tumors and depletes tumor-associated regulatory T cell levels in MC38 tumors. Results are shown as the number of regulatory T cells per 20 mg of (FIG. 6A) CT26 and (FIG. 6B) MC38 tumors in the absence (●) or the presence of the anti-CTLA-4 mAb (▴ for CT26 tumors and ▪ for MC38 tumors).

### DETAILED DESCRIPTION

Preliminary studies in mice have demonstrated that eosinophils can eliminate cancerous cells (Carretero et al. (2015) and Simson et al. (2007)). Clinical data have also previously shown that an increase in the blood levels of eosinophils in human patients treated with an anti-CTLA-4 mAb ipilimumab) is associated with a concomitant increase in the therapeutic activity of the therapeutic antibody used (Delyon et al. (2013)), supporting the importance of eosinophils in the treatment of cancer. However, as described herein and shown in the Example below, depleting, at least partially, eosinophils increased the therapeutic activity of anti-cancer immune-stimulating therapies. The present disclosure thus provides methods and therapeutic uses for depleting, at least partially, eosinophils to increase the therapeutic activity of an anti-cancer immune-stimulating agent and, in some embodiments, to treat or alleviate the symptoms of cancer. The present disclosure also provides methods for identifying potential therapeutic agents capable of increasing the therapeutic activity of an anti-cancer immune-stimulating agent and, in some embodiments, treating or alleviating the symptoms of cancer.

Interestingly, as described herein and shown in the Example below, administering anti-cancer immunotherapies was also shown to induce, in certain embodiments, a reduction in the number/level of tumoral regulatory T cells and such reduction was associated with a less effective anti-cancer therapy. The present disclosure thus also provides methods of identifying responder subjects to an anti-cancer immune-stimulating agents and of treating, using personalized medicine, subjects with an anti-cancer immune-stimulating agents.

i) Definitions

Agonist. This term, as used herein, refers to an agent that upregulates (e.g., increases, potentiates or supplements) the biological activity of a receptor. The term “agonistic antibody” is used herein to refer to an antibody, an antibody derivative or an antibody fragment capable of activating the biological activity of a receptor usually found on an immune cell, but not on an eosinophil.

Antagonist. This term, as used herein, refers to an agent that downregulates (e.g., decreases, inhibits or abrogates) the biological activity of a receptor. The term “antagonistic antibody” is used herein to refer to an antibody, an antibody derivative or an antibody fragment capable of decreasing/inhibiting the biological activity of a receptor usually found on an eosinophil.

Antibody. In the present disclosure, the therapeutic agent can be an antibody. Naturally occurring antibodies or immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system have been defined by Kabat et al. (Kabat, E. A. et al., Sequences of Proteins of Immunological Interest, Fifth Edition, U.S. Department of Health and Human Services, U.S. Government Printing Office (1991)).

The antibodies disclosed herein can be polyclonal or monoclonal antibodies. The antibodies disclosed herein also include antibody fragments. As used herein, a “fragment” of an antibody is a portion of an antibody that is capable of specifically recognizing the same epitope as the full version of the antibody. Antibody fragments include, but are not limited to, the antibody light chain, single chain antibodies, Fv, Fab, Fab′ and F(ab′)2 fragments. Such fragments can be produced by enzymatic cleavage or by recombinant techniques. For instance, papain or pepsin cleavage can be used to generate Fab or F(ab′)2 fragments, respectively. Antibodies can also be produced in a variety of truncated forms using antibody genes in which one or more stop codons have been introduced upstream of the natural stop site. For example, a chimeric gene encoding the heavy chain of an F(ab′)2 fragment can be designed to include DNA sequences encoding the CH1 domain and hinge region of the heavy chain.

The antibody disclosed herein can be an antibody derivative, such as, for example, a chimeric antibody and a humanized antibody. As used herein, the term “chimeric antibody” refers to an immunoglobulin comprising two regions from two distinct animals. As used herein, the term “humanized antibody” refers to a subsection of a “chimeric antibody” and includes an immunoglobulin that comprises both a region derived from a human antibody or immunoglobulin and a region derived from a non-human antibody or immunoglobulin. The action of humanizing an antibody consists in substituting a portion of a non-human antibody with a corresponding portion of a human antibody. For example, a humanized antibody as used herein could comprise a non-human region variable region (such as a region derived from a murine antibody) capable of specifically recognizing its target and a human constant region derived from a human antibody. In another example, the humanized immunoglobulin can comprise a heavy chain and a light chain, wherein the light chain comprises a complementarity determining region derived from an antibody of non-human origin which binds its target and a framework region derived from a light chain of human origin, and the heavy chain comprises a complementarity determining region derived from an antibody of non-human origin which binds its target and a framework region derived from a heavy chain of human origin. Antibody fragments can also be humanized. For example, a humanized light chain comprising a light chain CDR (i.e. one or more CDRs) of non-human origin and a human light chain framework region. In another example, a humanized immunoglobulin heavy chain can comprise a heavy chain CDR (i.e., one or more CDRs) of non-human origin and a human heavy chain framework region. The CDRs can be derived from a non-human immunoglobulin.

In some embodiments (imaging for example), it may be advantageous to couple the antibody, antibody fragment or antibody derivative to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive materials include ¹²⁵I, ¹³¹I, ³⁵S or ³H. Alternatively, the antibody, antibody fragment or antibody derivative can be coupled to a chemotherapeutic agent; a toxin (e.g., an enzymatically active toxin of bacterial, fungal, plant, or animal origin, or fragments thereof); a radioactive isotope (i.e., a radioconjugate). Exemplary toxins include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

The antibody, antibody derivative and antibody fragment are specific for at least one antigen. As used in the context of the present disclosure, an antibody, an antibody derivative or an antibody fragment is specific for an antigen when it is able to discriminate specifically the antigen from other (related or unrelated antigens). In some embodiments, the antigen is present on various immune cells and as such, even though the antibody, its derivative or its target is specific for an antigen, they can bind with specificity to different immune cells. In alternative embodiments, the antigen is present on only one type of immune cells (eosinophils for example) or on only one subtype of immune cells (tumor-associated eosinophils for example) and as such, they can bind specifically to such type or subtype of immune cells.

Pharmaceutically effective amount or therapeutically effective amount. These expressions refer to an amount (dose) effective in mediating a therapeutic benefit to a subject (for example prevention, treatment and/or alleviation of symptoms of cancer). It is also to be understood herein that a “pharmaceutically effective amount” may be interpreted as an amount giving a desired therapeutic effect, either taken in one dose or in any dosage or route, taken alone or in combination with other therapeutic agents.

Prevention, treatment and alleviation of symptoms. These expressions refer to the ability of a method, a therapeutic agent or a combination of therapeutic agents to limit the development, progression and/or symptomology of a proliferation-associated disorder, such as cancer. Broadly, the prevention, treatment and/or alleviation of symptoms can encompass the reduction of proliferation of the cells (e.g., by reducing the total number of cells in an hyperproliferative state and/or by reducing the pace of proliferation of cells). Symptoms associated with cancer, but are not limited to: local symptoms which are associated with the site of the primary cancer (such as lumps or swelling (tumor), hemorrhage, ulceration and pain), metastatic symptoms which are associated to the spread of cancer to other locations in the body (such as enlarged lymph nodes, hepatomegaly, splenomegaly, pain, fracture of affected bones, and neurological symptoms) and systemic symptoms (such as weight loss, fatigue, excessive sweating, anemia and paraneoplastic phenomena).

ii) Anti-Cancer Immune Therapies

Eosinophil-depleting agents. The present disclosure provides eosinophil-depleting agents to be used either to increase the therapeutic activity of an anti-cancer immune-stimulating agent(s) and/or to treat or alleviate the symptoms of cancer in a subject. The expression “eosinophil-depleting agent” refers to a therapeutic agent or a combination of therapeutic agents capable of reducing the number of eosinophils upon administration to a subject. Eosinophils can be are easily identified by hematoxylin and eosin staining of histological sections due to the bright pink staining of their basic granules. The reduction or depletion can be total, e.g. no circulating eosinophil can be detected in the subject. Alternatively, the reduction or depletion of eosinophils can be partial, e.g. some circulating eosinophils can be detected in the subject. In an embodiment, a partial reduction or depletion of eosinophils can be achieve by reducing by at least 5%, 10%, 15%, 20%, 25%, 30%, 35%, 40%, 45%, 50%, 55%, 60%, 65%, 70%, 75%, 80%, 85%, 90%, 95%, 99% or more the level/number of (circulating or tumor-associated) eosinophils. In some embodiments, the reduction or depletion is observed in circulating eosinophils and/or in tumor-associated eosinophils. In a specific embodiment, the reduction or depletion is observed in circulating eosinophils. The reduction or depletion in eosinophils levels does not have to be permanent, it can be transient and preferably last during the therapeutic window of the anti-cancer immune stimulating agents.

Eosinophil-depleting agents are thus capable of reducing the number of (circulating and/or tumor-associated) eosinophils and, in some embodiment, depleting the number of (circulating and/or tumor-associated) eosinophils. Eosinophil-depleting agents are acellular components, e.g. they cannot be cells. However, eosinophil-depleting agents can be derived from a cell or not. Eosinophil-depleting agents can be a small molecule, a nucleic acid molecule, a peptide, a polypeptide or an antibody or any additional acellular component capable of reducing/depleting eosinophils.

In an embodiment, the eosinophil-depleting agents can be an antibody (including an antibody derivative or an antibody fragment) specific for a growth factor or a cytokine exhibiting its (activating) biological activity in eosinophils. The eosinophil-depleting agent can also be a soluble receptors of such growth factors and cytokines. In such embodiments, the eosinophil-depleting agents can act as a “sink” for the growth factor or the cytokine to ultimately favor the reduction in the levels of (circulating and/or tumor-associated) eosinophils. Alternatively, the eosinophil-depleting agent can be an antagonistic anti-growth factor receptor/anti-interleukin receptor antibody capable of limiting the interaction between the growth factor/interleukin and its corresponding ligand to ultimately favors the reduction in the levels of (circulating and/or tumor-associated) eosinophils.

In an embodiment in which the eosinophil-depleting agent is specific for a cytokine, the cytokine can be an interleukin and the eosinophil-depleting agent can be an anti-interleukin (monoclonal) antibody, an antagonistic anti-interleukin receptor (monoclonal) antibody or a soluble interleukin receptor. In another embodiment in which the cytokine is IL-5, the eosinophil-depleting agent can be an anti-IL-5 antibody (such as, for example, mepolizumab or reslizumab), an antagonistic anti-IL-5 receptor antibody and/or a soluble IL-5 receptor (e.g., a soluble IL-5RA, such as, for example benralizumab). In another embodiment when the cytokine is IL-4, the eosinophil-depleting agent can be an anti-IL-4 antibody, an antagonistic anti-IL-4 receptor antibody (such as, for example, an anti-IL-4RA antibody, e.g., dupilumab and AMG 317) and/or a soluble IL-4 receptor. In another embodiment when the cytokine is IL-13, the eosinophil-depleting agent can be an anti-IL-13 antibody, an antagonistic anti-IL-13 receptor antibody (such as, for example, an antibody capable of binding to one or both of IL-13Rα1 and IL-13Rα2) and/or a soluble IL-13 receptor (such as, for example, a soluble IL-13 receptor, e.g., a soluble IL-13RA2). In still another embodiment when the cytokine is IL-25, the eosinophil-depleting agent can be an anti-IL-25 antibody, an antagonistic anti-IL-25 receptor antibody and/or a soluble IL-25 receptor. In still another embodiment when the cytokine is IL-33, the eosinophil-depleting agent can be an anti-IL-33 antibody, an antagonistic anti-IL-33 receptor antibody (such as, for example, an anti-ST2 antibody) and/or a soluble IL-33 receptor (such as, for example, a soluble ST2). In still another embodiment when the cytokine is anti-thymic stromal lymphopoietin (TSLP), the eosinophil-depleting agent can be an anti-TSLP antibody, an antagonistic anti-TSLP receptor antibody and/or a soluble TSLP receptor.

In another embodiment in which the eosinophil-depleting agent specific for a cytokine, the cytokine can be a chemokine and the eosinophil-depleting agent can be an anti-chemokine (monoclonal) antibody, an antagonistic anti-chemokine receptor (monoclonal) antibody or a soluble chemokine receptor. In a further embodiment when the chemokine is anti-C-C motif chemokine receptor 3 (CCR3), the eosinophil-depleting agent can be an anti-CCR3 antibody, an antagonistic anti-CCR3 receptor antibody and/or a soluble CCR3 receptor.

In yet another embodiment in which the eosinophil-depleting agent is specific for a growth factor, the eosinophil-depleting agent can be an anti-growth factor (monoclonal) antibody, an antagonistic anti-growth factor receptor (monoclonal) antibody or a soluble growth factor receptor. In an embodiment, the growth factor is epidermal growth factor (EGF) and the eosinophil-depleting agent can be an anti-EGF antibody, an antagonistic anti-EGFR antibody (such as, for example, an anti-EGF-like module-containing mucin-like hormone receptor-like1 or EMR1 (monoclonal) antibody) or a soluble EGFR.

In another embodiment, the eosinophil-depleting agent can be an antibody (including an antibody variant and an antibody fragment) specific to a cell-surface protein or can be a soluble version of the cell-surface protein. In such embodiment, the binding of the antibody to the cell-surface protein or the presence of the soluble cell-surface protein prevents or favors interactions of the cell-surface protein with other polypeptides and ultimately causes the reduction in the levels of (circulating and/or tumor-associated) eosinophils. Alternatively, the antibody can favor the reduction/depletion of eosinophils by triggering antibody-dependent cellular cytotoxicity. For example, the cell-surface protein can be CD300a (also referred to as Inhibitory Receptor Protein 60 or IRp60) and the eosinophil-depleting agent can be an anti-CD300a antibody or a soluble CD300a. In yet another example, the cell-surface protein can be Sialic Acid Binding Ig Like Lectin 8 (also referred to as Siglec-8) and the eosinophil-depleting agent can be an anti-Siglec-8 antibody or a soluble Siglec-8. In still another example, the cell-surface protein can be CD48 and the eosinophil-depleting agent can be an anti-CD48 antibody or a soluble CD48.

In an embodiment, the eosinophil-depleting agent is an antibody to a prostaglandin, an antagonistic antibody to a prostaglandin receptor or can be a soluble version of a prostaglandin receptor. In such embodiment, the binding of the antibody to the prostaglandin or its receptor prevents interactions between the prostaglandin and its receptor and ultimately causes the reduction in the levels of (circulating and/or tumor-associated) eosinophils. For example, the prostaglandin can be a prostaglandin D2 and the eosinophil-depleting agent can be an anti-prostaglandin D2 antibody, an anti-prostaglandin D2 receptor antagonistic antibody (e.g., an anti-chemoattractant receptor-homologous molecule expressed on Th2 cells (CRTH2)) or a soluble prostangladin D2 receptor (e.g., soluble CRTH2). Alternatively, the eosinophil-depleting agent favors the reduction/depletion of eosinophils by triggering antibody-dependent cellular cytotoxicity. In such embodiment, the antibody can be an anti-chemoattractant receptor-homologous molecule expressed on Th2 cells (CRTH2).

When the eosinophil-depleting agent is an antibody, it can be designed to be specific to one polypeptide (e.g., monospecific) or having a plural specificity to more than one polypeptide as described herewith. For example, the antibody can be bispecific for two polypeptides as described herein, such as exhibiting specific towards both CD300a with CCR3, and, in such embodiment, be capable of causing the aggregation of both antigens.

The eosinophil-depleting agent can be a single type of antibody for a single eosinophil-depleting target or a combination of more than one antibody each specific for the same or a different eosinophil-depleting target(s). For example, the eosinophil-depleting agent can include one, two, three, four, five or more different antibodies which can be specific to one, two, three, four, five or more different eosinophil-depleting targets. In an embodiment, the eosinophil-depleting agent can include an anti-IL-25 (monoclonal) antibody, an anti-TSLP 25 (monoclonal) antibody and an anti-IL-33 25 (monoclonal) antibody.

In still another embodiment, the eosinophil-depleting agent can be, for example, a small molecule known to be able to reduce or deplete the levels of (circulating or tumor specific) eosinophils. For example, the eosinophil-depleting agent can be polyinosinic-polycytidylic acid, a compound known to activate natural killer cells, which ultimately causes a reduction in eosinophil recruitment to ultimately reduce or deplete the (circulating or tumor-associated) eosinophils.

As indicated above, the eosinophil-depleting agent can be used to increase the therapeutic activity of an anti-cancer immune-stimulating agent. In such embodiment, the eosinophil-depleting agent must be used or administered in such a way that a reduction in the (circulating or tumor-associated) eosinophil is observed during the therapeutic window of the anti-cancer immune stimulating agent (e.g., the anti-cancer immune-stimulating agent's therapeutic activity window is observed while there is a reduction or a depleting of eosinophils). In an embodiment, the eosinophil-depleting agent can be used or administered prior to the use or the administration of the anti-cancer immune stimulating agent. In an embodiment, the eosinophil-depleting agent can be used or administered concomitantly to the use or the administration of the anti-cancer immune stimulating agent. In an embodiment, the eosinophil-depleting agent can be used or administered after to the use or the administration of the anti-cancer immune stimulating agent. In an embodiment, the eosinophil-depleting agent can be used or administered prior to and concomitantly to the use or the administration of the anti-cancer immune stimulating agent. In an embodiment, the eosinophil-depleting agent can be used or administered prior to and after the use or the administration of the anti-cancer immune stimulating agent. In an embodiment, the eosinophil-depleting agent can be used or administered concomitantly and after to the use or the administration of the anti-cancer immune stimulating agent. In an embodiment, the eosinophil-depleting agent can be used or administered prior to, concomitantly and after the use or the administration of the anti-cancer immune stimulating agent.

Anti-cancer immune-stimulating agent. As used in the context of the present, the expression “anti-cancer immune-stimulating agent” refer to a therapeutic agent or a combination of therapeutic agents capable of stimulating the biological activity at least one non-eosinophil immune cell (lymphocytes such as, for example, B cells and T cells, macrophages, dendritic cells, natural killer cells, etc.) for stimulating the immune response against the cancer cells. In some embodiments, the anti-cancer immune-stimulating agent does not have the ability to stimulate the biological activity of eosinophils. In some embodiments, the increase in biological activity is observed in circulated and/or tumor associated immune cells. This increase in biological activity does not have to be permanent, it can be transient, and preferably substantially match the therapeutic window of the eosinophil-depleting agent.

In an embodiment, the anti-cancer immune-stimulating agent can be antagonistic specific for an immune check-point. As used herein, the term “immune check-point” refers to a cell surface protein, present on immune cells, whose activity need to be reduced, abolished or inhibited to increase the biological activity of the immune cell to ultimately stimulate the immune response. In such embodiment, the anti-cancer immune-stimulating agent can be an antagonistic (monoclonal) antibody for the immune check-point or a (neutralizing) antibody to the ligand of the immune check-point. For example, the immune check-point can be the cytotoxic T-lymphocyte associated protein 4 (CTLA-4) protein and the anti-cancer immune-stimulating agent can be an anti-CTLA-4 antibody (such as ipilimumab) or an anti-CTLA-4 ligand antibody. In another embodiment, the immune check-point can be the programmed cell death 1 (PD-1) protein and the anti-cancer immune-stimulating agent can be an anti-PD-1 antibody (such as, for example, pembrolizumab or nivolumab) or an anti-PD-1 ligand antibody (e.g., an anti-programmed death ligand 1 (PD-L1) antibody, such as, for example atezolizumab, avelumab or durvalumab). In still another example, the immune check-point can be the T-cell immunoglobulin and mucin-domain containing-3 (TIM-3) protein and the anti-cancer immune-stimulating agent can be an anti-TIM-3 antibody or an anti-TIM-3 ligand antibody. In yet another example, the immune check-point can be the lymphocyte-activation gene 3 (LAG-3) protein and the anti-cancer immune-stimulating agent can be an anti-LAG-3 antibody or an anti-LAG-3 ligand antibody. In another example, the immune check-point can be CD244 (also referred to as 2B4) and the anti-cancer immune-stimulating agent can be an anti-CD244 antibody or an anti-CD244 ligand antibody. In a further example, the immune check-point can be the T cell immunoreceptor with Ig and ITIM domains (TIGIT) protein and the anti-cancer immune-stimulating agent can be an anti-TIGIT antibody or an anti-TIGIT ligand antibody (such as, for example, an anti-CD155 antibody). In a further example, the immune check-point can be CD96 and the anti-cancer immune-stimulating agent can be an anti-CD96 antibody or an anti-CD96 ligand antibody (such as, for example, an anti-CD155 antibody). In still another example, the immune check-point can be V-domain Ig suppressor of T cell activation (VISTA) protein and the anti-cancer immune-stimulating agent can be an anti-VISTA antibody or an anti-VISTA ligand antibody. In yet another example, the immune check-point can be CD112R and the anti-cancer immune-stimulating agent can be an anti-CD112R antibody or an anti-CD112R ligand (such as, for example, an anti-CD112 antibody).

In another embodiment, the anti-cancer immune-stimulating agent can be an agonistic antibody for a cell-surface protein (including a cell receptor) expressed on the surface of an immune cell capable whose activity is increased, upregulated or augmented to increase the biological activity of an immune cell and ultimately stimulate the immune response. In such embodiments, the antibodies are characterized as agonistic as they are capable of increasing the biological activity to the cell-surface protein. In an example, the cell surface protein can be a TNF receptor superfamily member 4 protein (referred to as TNFRSF4 or OX40) and the anti-cancer immune-stimulating agent can be an anti-TNFRSF4 antibody. In still another example, the cell surface protein can be a TNF receptor superfamily member 9 protein (referred to as TNFRSF9 or CD137 or 41BB) and the anti-cancer immune-stimulating agent can be an anti-TNFRSF9 antibody. In still another example, the cell surface protein can be a TNF receptor superfamily member 18 protein (referred to as TNFRSF18 or GITR) and the anti-cancer immune-stimulating agent can be an anti-TNFRSF18 antibody. In still another example, the cell surface protein can be a CD27 protein and the anti-cancer immune-stimulating agent can be an anti-CD27 antibody. In still another example, cell surface protein can be a CD28 protein and the anti-cancer immune-stimulating agent can be an anti-CD28 antibody. In yet another example, the cell surface protein can be a CD40 protein and the anti-cancer immune-stimulating agent can be an anti-CD40 antibody.

In another embodiment, the anti-cancer immune-stimulating agent can be an antagonistic antibody for a (cell-surface protein) (including a cell receptor) or soluble protein (including a cell receptor ligand) expressed by tumor cells and/or immune cells whose activity need to be reduced, abolished or inhibited to increase the biological activity of immune cell against tumor cells. In an example, the cell surface protein can be a CD47 protein and the anti-cancer immune-stimulating agent can be an anti-CD47 antibody. In another example, the cell surface protein can be a macrophage colony-stimulating factor receptor (M-CSFR also known as CSF1R) and the anti-cancer immune-stimulating agent can be an anti-M-CSFR antibody. In still another example, the cell surface and soluble protein can be CD73 and the anti-cancer immune-stimulating agent can be an anti-CD73 antibody. In yet another example, the cell surface protein can be CD39 and the immune-stimulating agent can be an anti-CD39 antibody. In still another example, the soluble protein can be CCL2 and the immune-stimulating agent can be an anti-CCL2 antibody.

When the anti-cancer immune-stimulating agent is an antibody, it can be designed to be specific to one polypeptide (e.g., monospecific) or having a plural specificity to more than one polypeptide as described herewith. The anti-cancer immune-stimulating agent can be a single type of antibody for a single anti-cancer immune-stimulating target or a combination of more than one antibody each specific for the same or a different anti-cancer immune-stimulating target(s). For example, the anti-cancer immune-stimulating agent can include one, two, three, four, five or more different antibodies which can be specific to one, two, three, four, five or more different immune-stimulating targets.

In additional embodiments, the anti-cancer immune-stimulating agent can be a viral infection (e.g., oncolytic viral infection, for example, by talimogene laherparepvec), an adoptive cellular therapy (e.g., an adoptive cell therapy with chimeric antigen receptor (CAR)-expressing T cells, an adoptive cell therapy with transgenic T cell receptor (TCR)-expressing T cells, an adoptive cell therapy with autologous tumor-infiltrating T cells and/or an adoptive cell therapy with allogeneic natural killer cells), a small molecule (e.g., adenosine receptor A2A antagonist, indoleamine 2,3-dioxygenase (IDO) inhibitors, tryptophan-2,3-dioxygenase (TDO) inhibitors, arginase 1 inhibitors), tumor vaccines (e.g., comprising tumor cells, antigen-presenting cells and/or mutated tumor antigenic peptides), agonists to Toll-like receptors, agonists to STING (stimulator of interferon genes), anti-transforming growth factor-β antibodies and/or bi-specific antibodies that redirect natural killer cell or T cell cytotoxicity to defined tumor antigens.

In some embodiments, the anti-cancer immune-stimulating agent has the ability to substantially maintain the level or number of tumor-associated regulatory T (Treg) cells and lacks the ability to substantially decrease the level or number of tumor associated Treg cells. In an embodiment, the anti-cancer immune-stimulating agent has the ability to maintain the level or the number of tumor associated Treg cells to at least 50%, 55%, 60%, 65%, 70%, 75%, 80%, 85%, 90%, 95%, 99% or more. As such, the present disclosure also includes determining if the anti-cancer immune-stimulating agent does substantially maintain the tumoral Treg levels and if so, administering or using the anti-cancer immune-stimulating agent (alone or in combination with the eosinophil-depleting agent) in subjects in which the anti-cancer immune-stimulating agent does substantially maintain the tumoral Treg levels.

The eosinophil-depleting agent has the ability to provide or increase the activity of the anti-cancer immune-stimulating agent. In an embodiment, the anti-cancer immune-stimulating agent has little to no therapeutic activity in the absence of the eosinophil-depleting agent. In another embodiment, the activity of the anti-cancer immune-stimulating agent is increased by at least 1%, 5%, 10%, 15%, 20%, 25%, 50% or more in the presence of the eosinophil-depleting agent when compared to the activity of the anti-cancer agent administered or used in the absence of eosinophil-depleting agent.

The eosinophil-depleting agent can also be used in combination with the anti-cancer immune-stimulating agent for preventing the reoccurrence of a previous cancer, treating an existing cancer and/or alleviating the symptoms associated with a cancer. The cancer can be, for example, a lung cancer (such as, for example, a non-small-cell lung cancer or a small-cell cancer), a breast cancer, a liver cancer (such as, for example, an hepatocellular carcinoma), a kidney/renal cancer (such as, for example, a renal cell carcinoma), a stomach cancer, a colorectal cancer, a head and neck tumor, an ovarian cancer, a pancreatic cancer (such as, for example, a pancreatic ductal adenocarcinoma), a bladder cancer, a skin cancer (such as, for example, a squamous cell carcinoma, a basal cell carcinoma, a Merkel cell carcinoma or an uveal melanoma), an esophagus cancer, a fallopian tube cancer, a genitourinary tract cancer (such as, for example, a transitional cell carcinoma or an endometrioid carcinoma), a prostate cancer (such as, for example, an hormone refractory prostate cancer), a stomach cancer, a nasopharyngeal cancer (such as, for example, a nasopharyngeal carcinoma), a peritoneal cancer, an adrenal gland cancer, an anal cancer, a thyroid cancer (such as, for example, an anaplastic thyroid cancer), a biliary cancer (such as, for example, cholangiocarcinoma), a gastro-intestinal cancer, a mouth cancer, a nervous system cancer, a penis tumor and/or a thymic cancer. The cancer can be a melanoma, a sarcoma, a mesothelioma, a glioblastoma, a lymphoma (such as, for example, a B-cell lymphoma (including diffuse large B-cell lymphoma), Hodgkins disease, a non-Hodgkin lymphoma, a multiple myeloma, a follicle center lymphoma, a peripheral T-cell lymphoma, a primary mediastinal large B-cell lymphoma or a myelodysplastic syndrome), a leukemia (such as, for example, an acute myelogenous leukemia, a chronic lymphocytic leukemia or a chronic myelocytic leukemia), a glioma and/or a melanoma. The cancer can be a stage I cancer, a stage II cancer, a stage Ill cancer or a stage VII cancer. The cancer can be a metastatic cancer. The cancer can be an hormone-sensitive or an hormone-refractory cancer.

The eosinophil-depleting agents and the anti-cancer immune-stimulating agents can be used/administered in various subjects, including, both not limited to, mammals such as humans. The eosinophil-depleting agents and the anti-cancer immune-stimulating agents can be used/administered to subjects having a solid tumor or a liquid tumor.

iii) Pharmaceutical Compositions and Combinations for Treating or Alleviating the Symptoms of Cancer

The eosinophil-depleting agents and the anti-cancer immune-stimulating agents can be provided as pharmaceutical compositions. As used herein, “pharmaceutical composition” means therapeutically effective amounts (dose) of the therapeutic agent together with pharmaceutically acceptable diluents, preservatives, solubilizers, emulsifiers, adjuvants and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, and detergents (e.g., Tween 20™, Tween 80™ Pluronic F68™, bile acid salts). The pharmaceutical composition can comprise pharmaceutically acceptable solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines).

Suitable methods of administering such therapeutic agents are available and well known to those of skill in the art, and, although more than one route can be used to administer a particular composition, a particular route can often provide a more immediate and more effective reaction than another route. The therapeutic agents disclosed herein may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the subject. The effective dosage is chosen from the range of 0.01 mg to 100 mg per kg of body weight per administration. Alternatively, the dosage in the range of 1 to 1000 mg, preferably 5 to 50 mg per patient may be chosen. When the therapeutic agents are antibodies, they are preferably administered parenterally and can be provided in a dry solid presentation for reconstitution (prior to administration) or in a ready-to-use presentation.

Administration is by any of the routes normally used for introducing a molecule into ultimate contact with blood or tumor cells. The therapeutic agents described herein can be administered in any suitable manner, preferably with the pharmaceutically acceptable carriers or excipients. The terms “pharmaceutically acceptable carrier”, “excipients” and “adjuvant” and “physiologically acceptable vehicle” and the like are to be understood as referring to an acceptable carrier or adjuvant that may be administered to a patient, together with a compound of this invention, and which does not destroy the pharmacological activity thereof. Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

The eosinophil-depleting agent can be provided in combination with the anti-cancer immune stimulating agent. In such embodiment, the eosinophil-depleting agent can be provided in the same pharmaceutical composition as the anti-cancer immune stimulating agent or both agents can be provided in different (discrete) pharmaceutical compositions.

The present disclosure also provides kits comprising the pharmaceutical compositions or the pharmaceutical combinations described herein as well as instructions on how to use or administered the therapeutic agents.

iv) Identification of Therapeutic Agents for Increasing the Therapeutic Activity of an Anti-Cancer Immune-Stimulating Agent

The present disclosure also provides a method for identifying the therapeutic agents which can be used to either increase the therapeutic activity of the anti-cancer immune-stimulating agent or can be used in combination with the anti-cancer immune stimulating agent to prevent, treat or alleviate the symptoms associated with cancer. The screening method is based on the fact that agents capable of reducing or depleting the (circulating or tumor-associated) eosinophils increase the therapeutic activity of the anti-cancer immune-stimulating agent and can be used in combination with the anti-cancer immune stimulating agent to prevent, treat or alleviate the symptoms associated with cancer.

The screening method first comprises determining the ability of the screened (test) agent to reduce or deplete the number of eosinophils and/or their biological activity. This step can be conducted in vitro (for example by determining the ability of the test agent to limit the biological activity of cell-cultured eosinophils or cause the apoptosis of eosinophils) or in vivo (for example by determining the ability of the test agent to reduce or eliminate circulating or tumor-associated eosinophils). When this determination step is conducted in vitro, it can be conducted with an eosinophil or an eosinophil precursor cell, a primary cell culture or a tumor cell culture. When this determination is made in vivo, it can be done in a non-human model, such as an animal model. If the determination occurs in a non-human model, then the model (such as a rodent) has a cancerous tumor is administered with the test agent. Various dosage and modes of administration may be used to fully characterize the test agent's ability to prevent, treat and/or alleviate the symptoms of a cancer. The number of eosinophils can be determined by various methods known in the art, including, but not limited to, cell sorting and immunological labeling.

The screening methods also comprises being provided with or, in some embodiments, determining the ability of a control agent to reduce or deplete the number of eosinophils and/or their biological activity. This step can include the same determination step as described above for the test agent and can be conducted in vitro or in vivo. The control agent can be an agent known to lack the ability to reduce or deplete eosinophils, a diluent used to prepare the test agent, etc.

In order to characterize the test agent as being useful (or not), a comparison between the results of the test agent and the control agent is undertaken. If the test agent is able to reduce the number of eosinophils more than the control agent, then the test agent is characterized as being useful for increasing the therapeutic activity of the anti-cancer immune-stimulating agent and/or for treating or alleviating the symptoms of the cancer subject (in combination with the anti-cancer immune-stimulating agent). In some embodiments, the screening method also allows to characterizing the test agent as not being useful for increasing the therapeutic activity of the anti-cancer immune-stimulating agent and/or for treating or alleviating the symptoms of the cancer subject (in combination with the anti-cancer immune-stimulating agent) when the test agent was not able to reduce/deplete more eosinophils than the control agent.

v) Personalized Medicine Approach for Anti-Cancer Immune-Stimulating Agents

The present disclosure also provides a method for identifying subjects who are more or less susceptible to respond to anti-cancer immune-stimulating agents (alone or in combination with the eosinophil-depleting agent). The identification method is based on the fact that the administration of anti-cancer immune-stimulating agents cause a substantial reduction in the levels of tumor-associated Treg cells. In return, this decrease in tumor-associated Treg cells is associated with a reduction in the therapeutic activity of the anti-cancer immune-stimulating agent. Therefore, the identification method is based on the determination of the levels of tumor-associated Treg cells (and more specifically the determination in the reduction of the levels of tumor-associated Treg cells) for identifying subjects susceptible or not to respond to the therapy.

The identification method first comprises determining the ability of the anti-cancer immune-stimulating thereby to reduce or deplete the number of tumor-associated Treg cells and/or their biological activity. This step can be conducted in vitro (for example by determining the ability of the anti-cancer immune-stimulating agent to limit the number or biological activity of Treg cells in a cultured tumor) or in vivo (for example by determining the ability of the anti-cancer immune-stimulating cell to reduce or eliminate tumor-associated Treg cells). When this determination step is conducted in vitro, it can be conducted in a tumor cell culture. When this determination is made in vivo, it can be done in a non-human model, such as an animal model. If the determination occurs in a non-human model, then the model (such as a rodent) has a cancerous tumor is administered with the anti-cancer immune-stimulating agent. Various dosage and modes of administration may be used to fully characterize the test agent's ability to prevent, treat and/or alleviate the symptoms of a cancer. The number of Treg cells can be determined by various methods known in the art, including, but not limited to, cell sorting and immunological labeling.

The identification methods also comprises being provided with or, in some embodiments, determining the ability of a control agent to reduce or deplete the number of Treg cells and/or their biological activity. This step can include the same determination step as described above and can be conducted in vitro or in vivo. The control agent can be an agent known to lack the ability to reduce or deplete tumor-associated Treg cells, a diluent used to prepare the test agent, or simply the number and/or biological activity of tumor-associated Treg cells prior to administering the anti-cancer immune-stimulating agent.

In order to characterize if the subject is a responder or not, a comparison between the results prior to and after the administration of the anti-cancer immune-stimulating agent is undertaken. If it is determined that the anti-cancer immune-stimulating agent is able to reduce the number of tumor-associated Treg cells, then the subject is characterized as not being a good responder (e.g., a poor responder). Alternatively, if it is determined that the anti-cancer immune-stimulating agent substantially maintains the number of tumor-associated Treg cells, then the subject is characterized as being a good responder.

In some embodiments, the method also comprises administering the anti-cancer immune-stimulating agent described herein (optionally in combination with the eosinophil-depleting agent described herein) to subjects having been identified as good responders.

The present invention will be more readily understood by referring to the following examples which are given to illustrate the invention rather than to limit its scope.

### EXAMPLE

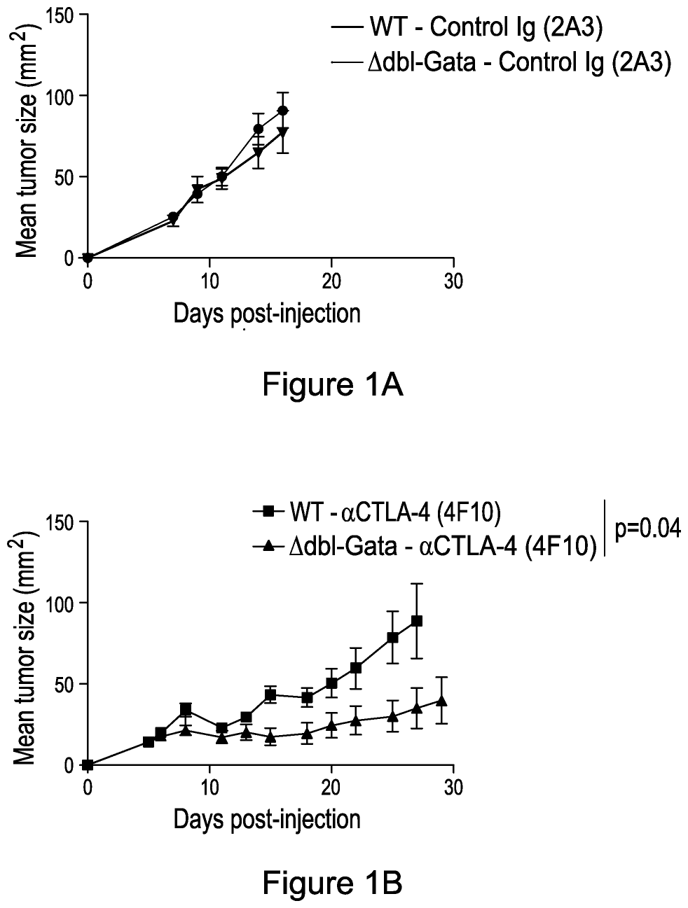

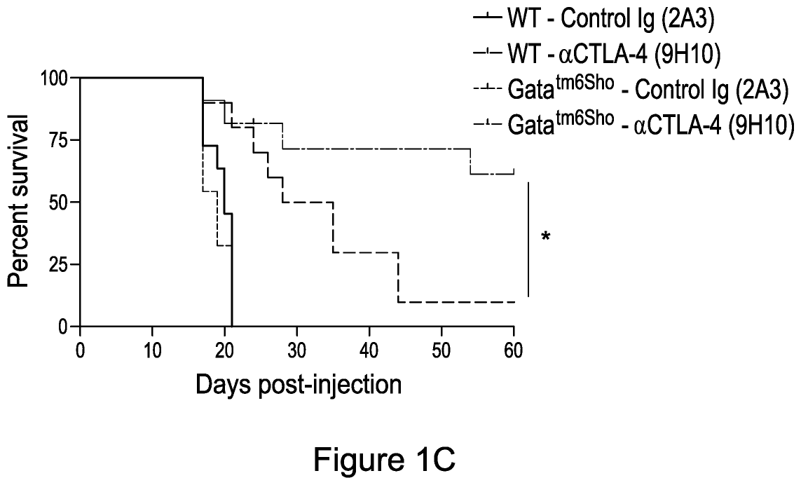

Anti-CTLA-4 therapy. Groups of 10 wild type (WT) and eosinophil-deficient (ΔdblGATA) syngeneic mice were injected subcutaneously (s.c.) with 3.5×10⁵ MC38 tumor cells and treated at day 5, 8 and 12 with anti-CTLA-4 mAb (200 micrograms clone 4F10 purchased from BioXcell injected intraperitoneally (i.p) in PBS) or a control Ig. Control Ig (clone 2A3) was purchased from BioXcell (10 Technology Dr, West Lebanon, N.H., USA) and injected intraperioneally. Clone 2A3 is a rat IgG2a antibody that reacts against trinitrophenol, not expressed by mammals. Tumor sizes (length×width) were measured at indicated time-points (depicted by symbols) using a digital caliper. Mean tumor sizes with standard errors are shown. In an independent experiment, WT and eosinophil-deficient (Δdbl GATA) syngeneic mice (11 per group) were injected subcutaneously (s.c.) with 5×10⁵ CT26 mouse tumor cells and treated at day 7, 11 and 14 with anti-CTLA-4 mAb (200 micrograms clone 9H10 purchased from BioXcell injected intraperitoneally (i.p) in PBS) or a control Ig. Mice were euthanized when tumors exceeded 150 mm² or when tumors were ulcerated. P values (Mann-Whitney and Log-rank) are indicated on FIG. 1.

The results shown on FIG. 1 indicate that the therapeutic activity of the anti-CTLA-4 antibodies was increased in eosinophil-depleted animals. In contrast to treatment with a control Ig (FIG. 1A), treatment with an anti-CTLA-4 mAb (FIG. 1B) was significantly more effective at reducing MC38 tumor growth in eosinophil-deficient (dlb-GATA) mice than in wild type mice. This contrasts with the previous publications of Carretero et al. (2015) and Simson et al. (2007) which both showed the importance of the presence eosinophils for eliminating cancerous cells in mice. This also contrasts with the publication of Delyon et al. (2013) which showed an increase in the blood levels of eosinophils in human patients treated with an anti-CTL4-4 mAb (i.e. ipilimumab) as well as with a concomitant increase in the therapeutic activity of the anti-CTL4-4 mAb used.

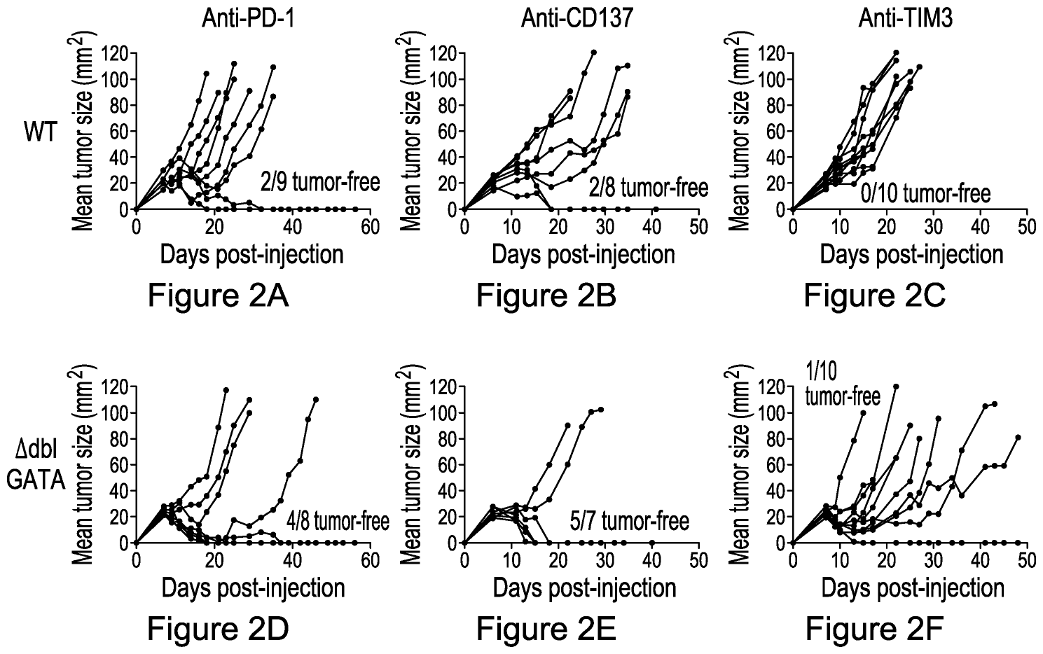

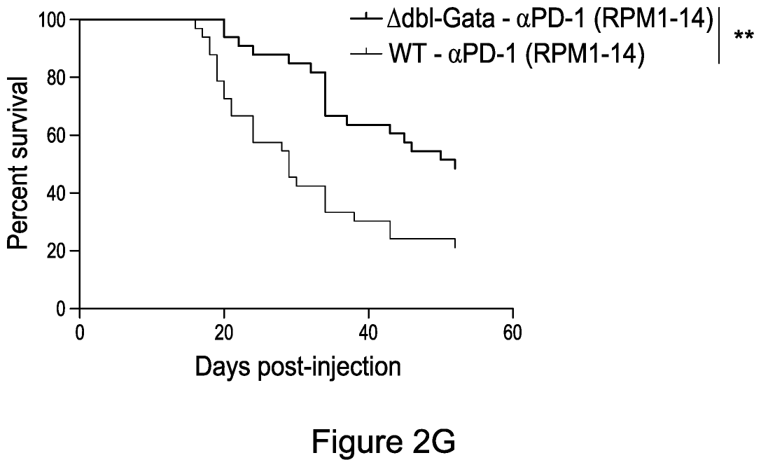

Anti-PD-1, anti-TIM3 and anti-CD37 therapy. As shown on FIG. 2, WT and eosinophil-deficient (Δdbl GATA) syngeneic mice were injected s.c. with 3.5×10⁵ MC38 tumor cells and treated at day 7, 11 and 14 with anti-PD-1 mAb (clone RMP1-14), anti-CD137 (clone 3H3) or anti-TIM3 mAb (clone RMT3-23). All mAbs were injected i.p. (200 micrograms each injection in PBS, all purchased from BioXCell). Tumor sizes (length x width) were measured at indicated time-points (depicted by symbols) using a digital caliper. Individual tumor growth curves are shown. Number of tumor-free mice at day 40 is indicated on FIG. 2. Anti-PD-1 mAb therapy (clone RMP1-14) of WT and eosinophil-deficient mice with subcutaneous MC38 tumors was evaluated in 4 independent experiments. Mice were euthanized when tumors exceeded 150 mm2 or when tumors were ulcerated. Survival of mice from the 4 experiments (33 mice per group) is depicted in FIG. 2. Log-rank P value less than 0.01 (**) is depicted on FIG. 2.

In agreement with the results shown on FIG. 1, the results shown on FIG. 2 indicate that the immunotherapies tested have increased therapeutic activity in eosinophil-deficient animals. As such, it was determined the activity of the immunotherapies could be improved by combining them with an eosinophil-depleting agent.

Anti-IL-5 combination therapy. Groups of 5 WT syngeneic mice were injected s.c. with 5×10⁵ CT26 cells and treated at day 7 and 11 with anti-CTLA-4 mAb (200 micrograms i.p. clone 9H10 purchased from BioXcell) and/or anti-IL-5 mAb (200 micrograms clone TRFKS). Tumor single cell suspensions were analyzed by flow cytometry at day 14 for tumor-specific CD8+ T cells using an MHC class I-gp70 tetramer (provided by the NIH tetramer core facility).

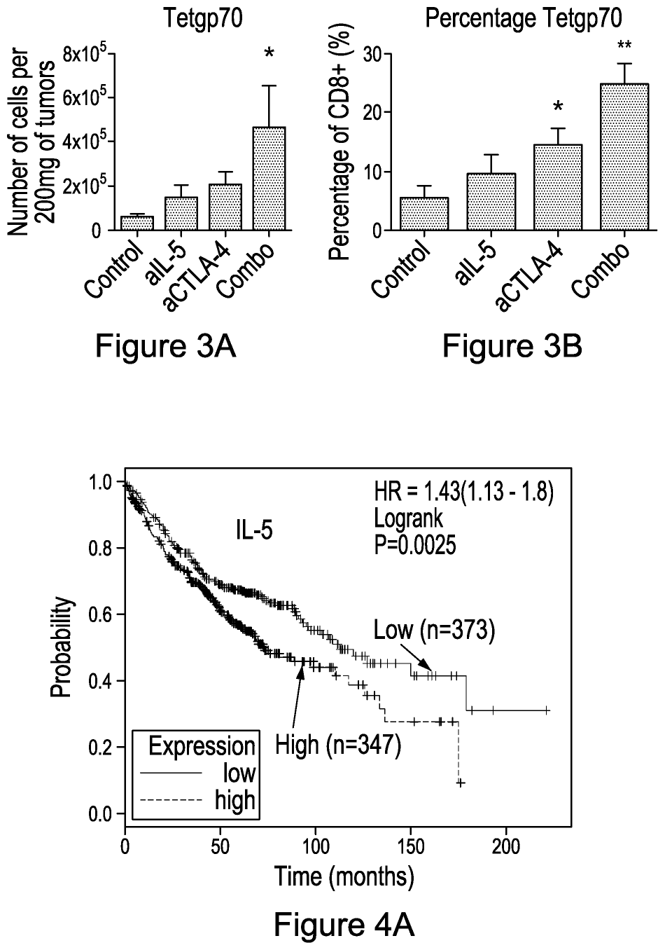

The results shown on FIG. 3 indicate that inhibiting the activity of IL-5, a potent signal for eosinophil to the tumor, increased the recruitment of anti-tumoral T cells in the tumors in the animals tested. These results also indicate that depleting eosinophils is advantageous since it increases the activity of the anti-CTLA-4 therapy.

Analysis of the expression of eosinophil-associated genes. Meta-analysis of microarray datasets of lung non-small cell adenocarcinomas was performed using www.kmplot.com software (Gyorffy B. et al., 2013). The correlation between overall survival and mRNA expression levels of IL-5 (207952_at), CCL-11 (also known as Eotaxin; 210133_at) and CCR3 (208304_at) was assessed by Log-rank analysis according to median gene expression.

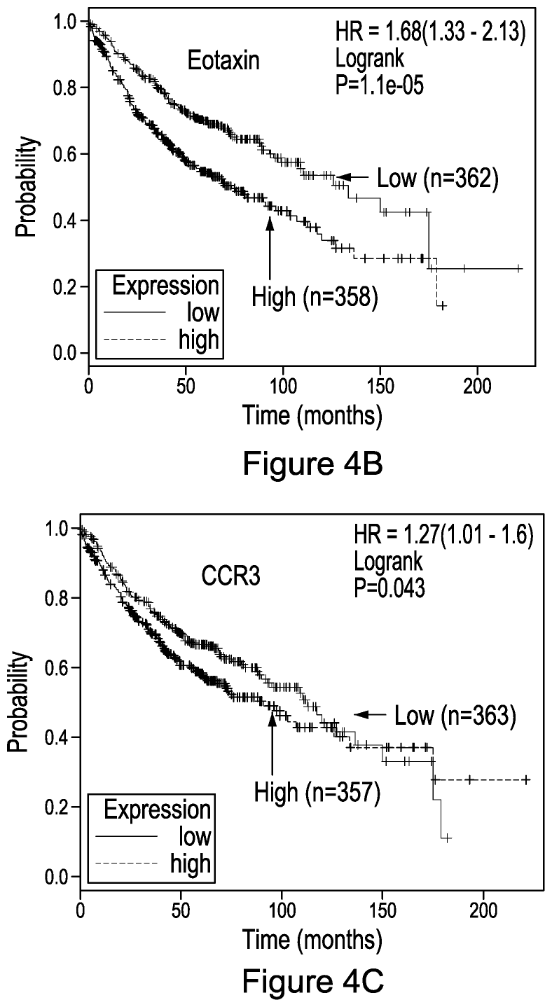

The results shown on FIG. 4 indicate that the high expression of IL-5, CCL-11 and CCR3 are associated with poor overall survival in lung non-small cell adenocarcinoma patients.

Anti-CTLA-4 therapy in CT26 and MC38 tumors. Groups of 10 wild type BALB/c and 10 ΔdblGATA BALB/c mice from The Jackson Laboratory were injected subcutaneously with 5×10⁵ CT26 mouse tumor cells (in 100 mL PBS) and treated at day 7, 11, 14 with anti-CTLA-4 antibody (100 micrograms clone 9H10 purchased from BioXcell) intraperitoneally diluted in 100 mL PBS. Tumor sizes (longest×shortest diameter) were measured using a digital caliper. Groups of 18 wild type C57/bl6 (from Charles River) and 18 ΔdblGATA C57/bl6 were injected subcutaneously with 2.5×10⁵ MC38 mouse tumor cells (in 100 mL PBS) and treated at day 7, 11, 14 with anti-CTLA-4 antibody (100 micrograms clone 9H10 purchased from BioXcell) intraperitoneally diluted in 100 mL PBS. Tumor sizes (longest x shortest diameter) were measured using a digital caliper.

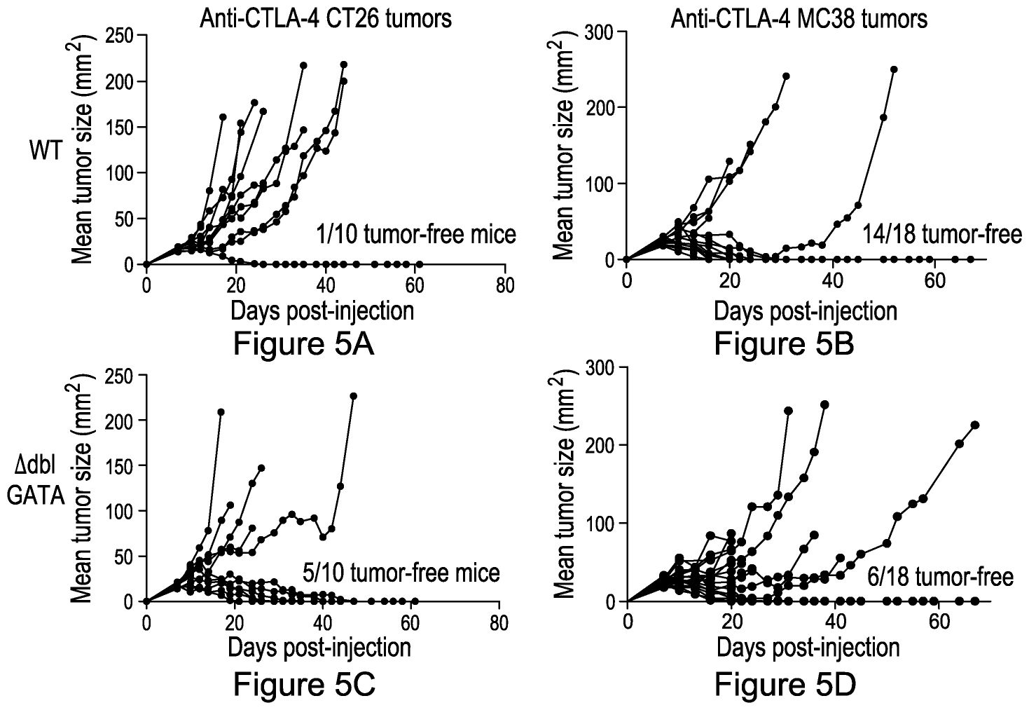

As shown on FIGS. 5, the anti-CTLA-4 therapy was useful in mice bearing CT26 tumors, but not MC38 tumors. To further investigate, an analysis of the tumoral immune cells of the tumors was conducted.

Treg analysis. Groups of wild type BALB/c and wild type C57/bl6 mice (from Charles River) were injected subcutaneously with 5×10⁵ CT26 mouse tumor cells (in 100 mL PBS) or 2.5×10⁵ MC38 mouse tumor cells (in 100 mL PBS), respectively and treated at day 7, 11, 14 with anti-CTLA-4 antibody (100 micrograms clone 9H10 purchased from BioXcell) intraperitoneally diluted in 100 mL PBS. At day 20 post-tumor cell injection, mice were euthanized, tumors excised, weighted, cut in pieces and exposed to a solution of collagenase type IV (and DNAse type I (Sigma). Lymphocytes were then purified using a Percoll solution of 40%-80% and single cell suspensions were stained with a panel of fluorochrome-conjugated antibodies consisting of CD4-APC (BD, 553051), CD8a-APC-H7 (BD, 560182), TCRb-PE (BD, 553172), and viability dye eF506 (eBioscience, 65-0866-14). Cell suspensions were than fixed with a Foxp3 Staining Buffer Set (eBioscience, 00-5523-00) to be than stained for Foxp3-Alexa488 (eBioscience, 53-5773-82). Samples were analyzed on LSRII Fortessa and data were analyzed with FlowJo software.

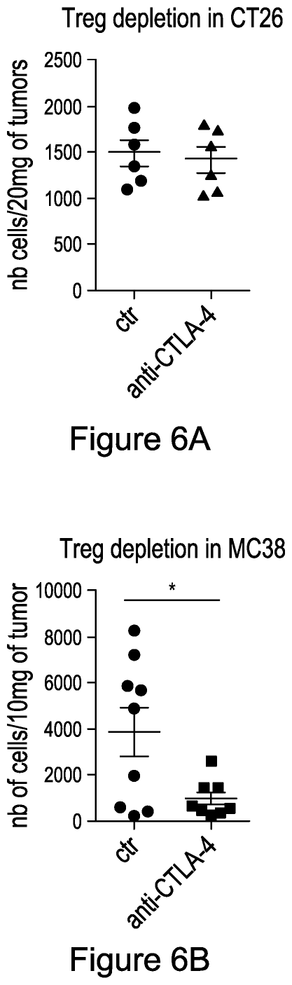

As shown on FIG. 6, when the anti-CTLA-4 therapy induced a reduction in the number of tumoral Treg. Anti-CTLA-4 therapy was shown less effective when it also reduced the number of tumoral Treg (FIG. 6B) than when it did not (FIG. 6A). The ability of decreasing the number of tumoral Treg is thus associated with a reduction in therapeutic activity for the anti-CTLA-4 therapy.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Carretero R, Sektioglu I M, Garbi N, Salgado O C, Beckhove P, Hammerling G J. Eosinophils orchestrate cancer rejection by normalizing tumor vessels and enhancing infiltration of CD8(+) T cells. Nat Immunol. 2015 June; 16(6):609-17.

Delyon J, Mateus C, Lefeuvre D, Lanoy E, Zitvogel L, Chaput N, Roy S, Eggermont A M, Routier E, Robert C. Experience in daily practice with ipilimumab for the treatment of patients with metastatic melanoma: an early increase in lymphocyte and eosinophil counts is associated with improved survival. Ann Oncol. 2013 June; 24(6):1697-703.

Gyorffy B, Surowiak P, Budczies J, Lanczky A. Online survival analysis software to assess the prognostic value of biomarkers using transcriptomic data in non-small-cell lung cancer. PLoS One. 2013 Dec. 18; 8(12):e82241.

Huang A C, Postow M A, Orlowski R J, Mick R, Bengsch B, Manne S, Xu W, Harmon S, Giles J R, Wenz B, Adamow M, Kuk D, Panageas K S, Carrera C, Wong P, Quagliarello F, Wubbenhorst B, D'Andrea K, Pauken K E, Herati R S, Staupe R P, Schenkel J M, McGettigan S, Kothari S, George S M, Vonderheide R H, Amaravadi R K, Karakousis G C, Schuchter L M, Xu X, Nathanson K L, Wolchok J D, Gangadhar T C, Wherry E J. T-cell invigoration to tumour burden ratio associated with anti-PD-1 response. Nature. 2017 May 4; 545(7652):60-65.

Luke J J, Flaherty K T, Ribas A, Long G V. Targeted agents and immunotherapies: optimizing outcomes in melanoma. Nat Rev Clin Oncol. 2017 Apr. 4. doi: 10.1038/nrclinonc.2017.43.

Seymour L, Bogaerts J, Perrone A, Ford R, Schwartz L H, Mandrekar S, Lin N U, Litière S, Dancey J, Chen A, Hodi F S, Therasse P, Hoekstra O S, Shankar L K, Wolchok J D, Ballinger M, Caramella C, de Vries E G; RECIST working group. iRECIST: guidelines for response criteria for use in trials testing immunotherapeutics. Lancet Oncol. 2017 March; 18(3):e143-e152.

Simson L, Ellyard J I, Dent L A, Matthaei K I, Rothenberg M E, Foster P S, Smyth M J, Parish C R. Regulation of carcinogenesis by IL-5 and CCL11: a potential role for eosinophils in tumor immune surveillance. J Immunol. 2007 Apr. 1; 178(7):4222-9.

1. A method of increasing the therapeutic activity of an anti-cancer immune-stimulating agent in subject having a tumor, the method comprises administrating an eosinophil-depleting therapeutic agent prior to, concomitantly or after having administered the anti-cancer immune-stimulating agent to the subject thereby increasing the therapeutic activity of the anti-cancer immune-stimulating agent, wherein, upon administration to the cancer subject:
i) the eosinophil-depleting agent is capable of at least partially depleting tumor-specific eosinophils, and
ii) the anti-cancer immune-stimulating agent lacks the ability to substantially reduce the number of tumor-associated regulatory T cells.

2. A method of treating or alleviating the symptoms of a cancer in a subject having a tumor, the method comprising administering an eosinophil-depleting agent and an anti-cancer immune-stimulating agent thereby treating or alleviating the symptoms of the cancer in the subject, wherein, upon administration to the subject:
i) the eosinophil-depleting agent is capable of at least partially depleting tumor-specific eosinophils, and
ii) the anti-cancer immune-stimulating agent lacks the ability to substantially reduce the number of tumor-associated regulatory T cells.

3. The method of claim 1, wherein the eosinophil-depleting agent is administered to the subject prior to or concomitantly with the anti-cancer stimulating agent.

4. The method of claim 1 further comprising determining if the anti-cancer immune-stimulating agent lacks the ability to substantially reduce the number of tumor-associated regulatory T cells in the subject.

5. The method of claim 1 to further comprising administering the eosinophil-depleting agent and/or the anti-cancer immune stimulating agent when the anti-cancer immune-stimulating agent has been determined to lack the ability to substantially reduce the number of tumor-associated regulatory T cells.

6. The method of claim 1, wherein the eosinophil-depleting agent is an antibody.

7. The method of claim 6, wherein the eosinophil-depleting agent is a monoclonal antibody.
